(19) United States
(12) Patent Application Publication
(10) Pub. No.: US 2021/0308102 A1
(43) Pub. Date: Oct. 7, 2021

(54) COMPOSITIONS AND METHODS FOR TREATMENT OF PRESBYOPIA

(71) Applicant: Seinda Pharmaceutical Guangzhou Corporation, Guangzhou (CN)

(72) Inventors: Hui Ouyang, San Diego, CA (US); Min Xia, San Diego, CA (US)

(21) Appl. No.: 17/266,813

(22) PCT Filed: Aug. 8, 2019
(86) PCT No.: PCT/US2019/045741

§ 371 Date: Feb. 8, 2021

Related U.S. Application Data
(60) Provisional application No. 62/715,915, filed on Aug. 8, 2018.

(51) Int. Cl. A61K 31/407; A61K 31/165; A61K 31/661; A61K 9/00; A61K 31/437; A61K 31/222; A61K 31/498; A61K 31/4168; A61P 27/06

(52) U.S. Cl. CPC A61K 31/407; A61K 31/165; A61K 31/661; A61K 9/0048; A61P 27/06; A61K 31/222; A61K 31/498; A61K 31/4168; A61K 31/437

(57) ABSTRACT

The present disclosure is directed to compositions and methods for treating presbyopia. The compositions can include a cholinesterase inhibitor, such as neostigmine, echothiophate, diisopropyl fluorophosphates, or physostigmine, and/or a mitoic agent. The compositions can act alone or synergistically, for example, to improve the accommodative and focusing ability of the eye while minimizing the side effects from each compound.

### CROSS-REFERENCE TO RELATED APPLICATIONS

This application claims the benefit of U.S. Provisional Patent Application Ser. No. 62/715,915, filed on Aug. 8, 2018. The disclosure of the prior application is considered part of and is incorporated by reference in the disclosure of this application.

### TECHNICAL FIELD

This document relates generally to treating presbyopia. More specifically, this document relates to the preparation and application of pharmaceutical composition to treat presbyopia.

### BACKGROUND

A normal eye has a balanced refractive power and axial length. It is a passive process without any active muscular contraction of a normal eye to see distant objects sharply. However, an active process that requires an active muscular increase in the refractive power of a normal eye is used to see near objects sharply. A presbyopic eye can have either a shorter axial length or a weaker refractive power and hence needs an active muscular mechanism to focus on near objects.

Currently, the most common way to correct presbyopia is by using reading glasses or bifocal glasses. Several surgical treatments have also been devised for the treatment of presbyopia including special intraocular lenses, laser reshaping of the cornea, and scleral expansors. Exercises have been proposed as a way to delay the onset of presbyopia. Pharmacological treatments for presbyopia have also been proposed.

### SUMMARY

Although some molecules have been disclosed for the medical treatment of presbyopia, clinically effective preparations suitable for use by the general public without unreasonable side effects have not previously been described.

Compositions and methods for treating presbyopia are disclosed herein. The compositions of the present invention include a cholinesterase inhibitor and/or a miotic agent. Examples of cholinesterase inhibitor include physostigmine, nostigmine, pyridostigmine, galantamine, donepezil, echothiophate, diisopropyl fluorophosphates; Examples of miotic agent include alpha-1 receptor antagonist, alpha-2 receptor agonist, β-adrenergic receptor antagonist, nicotine receptor agonist, adenosine receptor antagonist, antipsychotics, anti-emetics, cannabinoids, monoamine oxidase (MAO) inhibitors, EP1, EP4, and FP receptors agonists, and others.

In some embodiments, compositions of the described herein can unexpectedly act synergistically to correct presbyopic vision. In some embodiments, compositions described herein can also be used to potentiate or to enhance interventions that retard, reverse or modify the aging process of the crystalline lens and its surrounding tissues. In some embodiments, a composition described herein is administered in a pharmaceutically acceptable ophthalmic formulation. In some embodiments, a composition described herein is administered topically by application of the formulation to the eye in a non-irritating sterile solution or suspension. In some embodiments, a formulation is at a pH compatible with the eye. More particularly, in accordance with the present disclosure, a cholinesterase inhibitor may be selected to act synergistically with a miotic agent to treat presbyopia.

In one aspect, provided herein can be a method, the method including administering to an eye of a subject, during a treatment period, a cholinesterase inhibitor. In some embodiments, the method can further include administering to the eye of the subject, during the treatment period, a miotic agent. Also provided herein is a method, the method including administering to an eye of a subject, during a treatment period, a miotic agent. In some embodiments, the method can further include administering to the eye of the subject, during the treatment period, a cholinesterase inhibitor.

Implementations can include one or more of the following features. The cholinesterase inhibitor can be selected from the group consisting of physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphates, pharmaceutically acceptable salts thereof, and combinations thereof. The cholinesterase inhibitor can be selected from the group consisting of physostigmine, neostigmine, demecarium bromide, diisopropyl flurophosphate, pharmaceutically acceptable salts thereof, and combinations thereof. The cholinesterase inhibitor can include physostigmine or a pharmaceutically acceptable salt thereof. The cholinesterase inhibitor can include neostigmine or a pharmaceutically acceptable salt thereof. The cholinesterase inhibitor can include demecarium bromide. The cholinesterase inhibitor can include diisopropyl flurophosphate. The cholinesterase inhibitor can be administered at a concentration of about 0.01% to about 2.0% (w/v). The cholinesterase inhibitor can be administered at a concentration of about 0.05% to about 1.50% (w/v). The cholinesterase inhibitor can be administered at a concentration of about 0.05% (w/v). The cholinesterase inhibitor can be administered at a concentration of about 0.25% (w/v). The cholinesterase inhibitor can be administered at a concentration of about 0.75% (w/v). The cholinesterase inhibitor can be administered at a concentration of about 0.083% (w/v). The cholinesterase inhibitor can be administered at a concentration of about 1.0% (w/v). The cholinesterase inhibitor can be administered at a concentration of about 1.5% (w/v). The miotic agent can be selected from the group consisting of muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof. The miotic agent includes a muscarinic receptor agonist. The muscarinic receptor agonist can be selected from the group consisting of choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and combinations thereof. The miotic agent can include an alpha-1 adrenergic receptor antagonist. The alpha-1 adrenergic receptor antagonist can be selected from the group consisting of phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYKI-12743, GYKI-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof. The miotic agent can include an alpha-2 adrenergic receptor agonist. The alpha-2 adrenergic receptor agonist can be selected from the group consisting of apraclonidine, brimonidine, clonidine, mivazerol, naphazoline, oxymetazoline, tetrahydrozoline, guanfacine, guanabenz, guanoxabenz, guanethidine, xylazine, tizanidine, medetomidine, methyldopa, methylnorepinephrine, moxonidine, rilmenidine, fadolmidine, dexmedetomidine, amitraz, cannabivarin, detomidine, dihydroergotamine, dipivefrine, dopamine, ephedrine, ergotamine, esproquin, etilefrine, eEthylnorepinephrine, 6-fluoronorepinephrine, levonordefrin, lofexidine, naphazoline, 4-NEMD, (R)-3-nitrobiphenyline, norepinephrine, phenylpropanolamine, piperoxan, pseudoephedrine, rilmenidine, romifidine, talipexole, tetrahydrozoline, tolonidine, xylometazoline, a pharmaceutically acceptable salt thereof, and combinations thereof. The miotic agent can be administered at a concentration of about 0.01% to about 2.0% (w/v). The miotic agent can be administered at a concentration of about 0.05% to about 1.50% (w/v). The eye of the subject can be presbyoptic or can be at risk for developing presbyopia. The amount of the cholinesterase inhibitor, miotic agent, or combination thereof can be sufficient to inhibit, slow, or prevent progression of presbyopia in the eye. The amount of the cholinesterase inhibitor, miotic agent, or combination thereof can be sufficient to maintain a pupillary dilation of at least about 2.0 mm. The amount of the cholinesterase inhibitor, miotic agent, or combination thereof can be sufficient to maintain a pupillary dilation of at least about 2.5 mm. The amount of the cholinesterase inhibitor, miotic agent, or combination thereof can be sufficient to maintain a pupillary dilation of at least about 3.0 mm. The amount of the cholinesterase inhibitor, miotic agent, or combination thereof can be sufficient to maintain a pupillary dilation of at least about 3.5 mm. The amount of the cholinesterase inhibitor, miotic agent, or combination thereof can be sufficient to achieve a Log MAR visual acuity of less than about 0.3. The amount of the cholinesterase inhibitor, miotic agent, or combination thereof can be sufficient to achieve a Log MAR visual acuity of less than about 0.28. The amount of the cholinesterase inhibitor, miotic agent, or combination thereof can be sufficient to achieve a Log MAR visual acuity of less than about 0.25. The amount of the cholinesterase inhibitor, miotic agent, or combination thereof can be sufficient to achieve a Log MAR visual acuity of less than about 0.20. The Log MAR visual acuity can be assessed using a distance corrected near visual acuity (DCNVA) at 40 cm. The cholinesterase inhibitor and miotic agent can be administered concurrently. The cholinesterase inhibitor and miotic agent can be administered concurrently as separate agents. The cholinesterase inhibitor and miotic agent can be administered concurrently as a single formulation. The cholinesterase inhibitor and miotic agent can be administered sequentially. The cholinesterase inhibitor can be administered before the miotic agent. The cholinesterase inhibitor can be administered after the miotic agent. The treatment period can be from about 1 day to about 60 months. The cholinesterase inhibitor can be administered from 1-6 times per day during the treatment period. The cholinesterase inhibitor can be administered in an application in an amount of about 30 μL to about 80 μL per application. The miotic agent can be administered from 1-6 times per day during the treatment period. The miotic agent can be administered in an application in an amount of about 30 μL to about 80 μL per application. The method can further include treating the eye with an ocular device during at least a portion of the treatment period. The ocular device can be a lens or an implant. The ocular device can be a corrective lens. The method can further include correcting vision in the eye with a corrective lens during the treatment period. The need of the subject for correcting vision in the eye with a corrective lens can be decreased during the treatment period. The need of the subject for correcting vision in the eye with a corrective lens can be eliminated during the treatment period.

Also provided herein is a composition including a cholinesterase inhibitor and a pharmaceutically acceptable carrier. In some embodiments, the composition can further include a miotic agent. Also provided herein is a composition including a miotic agent and a pharmaceutically acceptable carrier. In some embodiments, the composition can further include a cholinesterase inhibitor.

Implementations can have one or more of the following features. The cholinesterase inhibitor can be selected from the group consisting of physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphates, pharmaceutically acceptable salts thereof, and combinations thereof. The cholinesterase inhibitor can be selected from the group consisting of physostigmine, neostigmine, demecarium bromide, diisopropyl flurophosphate, pharmaceutically acceptable salts thereof, and combinations thereof. The cholinesterase inhibitor can include physostigmine or a pharmaceutically acceptable salt thereof. The cholinesterase inhibitor can include neostigmine or a pharmaceutically acceptable salt thereof. The cholinesterase inhibitor can include demecarium bromide. The cholinesterase inhibitor can include diisopropyl flurophosphate. The cholinesterase inhibitor can be present at a concentration of about 0.01% to about 2.0% (w/v). The cholinesterase inhibitor can be present at a concentration of about 0.05% to about 1.50% (w/v). The cholinesterase inhibitor can be present at a concentration of about 0.05% (w/v). The cholinesterase inhibitor can be present at a concentration of about 0.25% (w/v). The cholinesterase inhibitor can be present at a concentration of about 0.75% (w/v). The cholinesterase inhibitor can be present at a concentration of about 0.083% (w/v). The cholinesterase inhibitor can be present at a concentration of about 1.0% (w/v). The cholinesterase inhibitor can be present at a concentration of about 1.5% (w/v). The miotic agent can be selected from the group consisting of muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof. The miotic agent can include a muscarinic receptor agonist. The muscarinic receptor agonist can be selected from the group consisting of choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and combinations thereof. The miotic agent can include an alpha-1 adrenergic receptor antagonist. The alpha-1 adrenergic receptor antagonist can be selected from the group consisting of phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYM-12743, GYKI-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof. The miotic agent can include an alpha-2 adrenergic receptor agonist. The alpha-2 adrenergic receptor agonist can be selected from the group consisting of apraclonidine, brimonidine, clonidine, mivazerol, naphazoline, oxymetazoline, tetrahydrozoline, guanfacine, guanabenz, guanoxabenz, guanethidine, xylazine, tizanidine, medetomidine, methyldopa, methylnorepinephrine, moxonidine, rilmenidine, fadolmidine, dexmedetomidine, amitraz, cannabivarin, detomidine, dihydroergotamine, dipivefrine, dopamine, ephedrine, ergotamine, esproquin, etilefrine, eEthylnorepinephrine, 6-fluoronorepinephrine, levonordefrin, lofexidine, naphazoline, 4-NEMD, (R)-3-nitrobiphenyline, norepinephrine, phenylpropanolamine, piperoxan, pseudoephedrine, rilmenidine, romifidine, talipexole, tetrahydrozoline, tolonidine, xylometazoline, a pharmaceutically acceptable salt thereof, and combinations thereof. The miotic agent can be present at a concentration of about 0.01% to about 2.0% (w/v). The miotic agent can be present at a concentration of about 0.05% to about 1.50% (w/v). The composition can further include a salt. The salt can be present in a concentration of about 0.2% to about 0.4% (w/v). The salt can include sodium chloride, zinc sulfate, potassium chloride, or a combination thereof. The composition can further include a buffer. The buffer can be present in an amount of about 0.8% to about 1.2% (w/v). The buffer can include boric acid, citric acid, or a combination thereof. The composition can further include a preservative. The preservative can be present in an amount of about 0.08% to about 0.15% (w/v). The preservative can include benzalkonium chloride, edetate disodium, or a combination thereof. The pH of the composition can be about 6.5 to about 7.5. The composition can further include a viscosity modifier. The viscosity modifier can include methyl cellulose, carboxymethyl cellulose, hydroxypropylmethyl cellulose, glycerol, polyethylene glycol, or combinations thereof. The composition can further include an osmotic agent. The osmotic agent can include mannitol, sorbitol, or a combination thereof. The composition can further include a surfactant, a solubilizer, a wetting agent, liposomes, DMSO, or a combination thereof. The solubilizer can include a cyclodextrin. The surfactant can include an anionic surfactant, a nonionic surfactant, or a combination thereof. The composition can be a topical preparation. The composition can be a solution, a suspension, an emulsion, a gel, or a sustained release formulation. The composition can be an ophthalmic preparation.

Also provided herein is a method of treating presbyopia, the method including administering to an affected eye of a subject in need of such treatment a therapeutically effective amount of any one or more of the compositions provided herein.

Also provided herein is use of any one or more of the compositions provided herein for the manufacture of a medicament for the treatment of presbyopia.

Also provided herein is a kit including any one or more of the compositions provided herein.

Also provided herein is an ophthalmological composition to treat presbyopia including a cholinesterase inhibitor, which can be a reversible, non-reversible, or quasi-reversible cholinesterase inhibitor, and can be used alone or in combination with other pharmaceutical agent.

Implementations can include one or more of the following features. The cholinesterase inhibitor can be selected from the group consisting of physostigmine, neostigmine, caffeine, donepezil, echothiophate, diisopropyl fluorophosphates, demecarium.

Also provided herein is an ophthalmological composition to treat presbyopia including a miotic agent, which can be used alone or in combination with other pharmaceutical agent.

Implementations can include one or more of the following features. The miotic agent can be an alpha-1 adrenergic receptor antagonist, alpha-2 adrenergic receptor agonist, beta-adrenergic receptor antagonist, nicotine receptor agonist, antipsychotics, anti-emetics, cannabinoid, MAO inhibitor, EP1, EP4, and FP receptors agonists, or calcium channel modulator. The miotic agent can be alpha-1 adrenergic receptor antagonist. The mitoic agent can be selected from the group consisting of dapiprazole, thymoxamine, brimonidine, nicotine, apraclonidin, bunazosin, phentolamine, and corynathine.

Also provided herein is an ophthalmological composition to treat presbyopia including a cholinesterase inhibitor in combination with a mitoic agent, to act synergistically to increase the depth of the focus of the eye.

Implementations can include one or more of the following features. The cholinesterase inhibitor concentration can range from 0.001%-2%, and the miotic agent concentration can range from 0.001%-5%. The composition can further include a viscosity enhancer and a surfactant selected from the group consisting of an anionic surfactant, a nonionic surfactant, and a combination thereof. The viscosity enhancer can be carboxymethyl cellulose or hydroxypropylmethyl cellulose. The anionic surfactant can be selected from the group consisting of a gamma cyclodextrin, sulfobutylether β-cyclodextrin, sodium lauryl sulfate and sodium ester lauryl sulfate. The nonionic surfactant can be selected from the group consisting of a poloxamer, a polysorbate, sorbitan monolaurate, sorbitan monopalmitate, sorbitan monostearate, sorbitan monooleate, polyoxyl stearate, a polyoxyl alkyl, a cyclodextrin and derivatives thereof. The composition can further include sulfobutylether β-cyclodextrin, polyoxyl 40 stearate, or 2-hydroxypropyl beta-cyclodextrin. The composition can further include an antioxidant can be selected from the group consisting ascorbic acid, glutathione, acetylcysteine, monothioglycerol, metabisulfite salts, sulfurous acid salts (bisulfite), dithiotheitol (DTT), dithiobutylamine (DTBA), and combinations thereof. The composition can further include a chelating agent can be selected from the group consisting ethylenediaminetetraacetic acid (EDTA), calcium disodium EDTA, disodium EDTA, pentetic acid (DTPA), and combinations thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a plot of pupil size change over time after drug administration according to Example 2.

FIG. 2 is a plot of DCNVA (distance corrected near visual acuity) change over time after drug administration according to Example 2.

### DETAILED DESCRIPTION

As used herein, “a”, “an”, “the”, “at least one”, and “one or more” are used interchangeably.

The terms “comprises” and variations thereof do not have a limiting meaning where these terms appear in the description and claims.

The terms “treat(ment)” or “treating” are used herein to denote delaying the onset of, preventing, inhibiting, alleviating the effects of, or regressing a disease or a symptom thereof in a subject.

The terms “therapeutically effective amount” and “effective amount” as used herein, refer to an amount or concentration of a composition or treatment described herein, utilized for a period of time (including acute or chronic administration and periodic or continuous administration) that is effective within the context of its administration for causing an intended effect or physiological outcome. For example, a therapeutically effective amount of a muscarinic receptor agonist is an amount sufficient to inhibit, slow, or prevent progression of presbyopia in the eye. As another example, a therapeutically effective amount of a muscarinic receptor agonist is an amount sufficient to inhibit or slow shrinkage in the axial length of the eye.

The term “subject” is used throughout the specification to describe an animal, human or non-human, to whom treatment according to the methods of the present disclosure is provided. Human and veterinary applications are anticipated by the present disclosure. The term includes but is not limited to birds, reptiles, amphibians, and mammals, e.g., humans, other primates, pigs, rodents, such as mice and rats, rabbits, guinea pigs, hamsters, horses, cows, cats, dogs, sheep, chickens and goats. In some embodiments, the subjects are humans, chickens, or mice. In some aspects, the subject is a human. Both pediatric and adult subjects are included. For example, in any of the methods described herein, the subject can be at least 6 months old (e.g., 6 months or older, 12 months or older, 18 months or older, 2 years or older, 4 years or older, 6 years or older, 10 years or older, 13 years or older, 16 years or older, 18 years or older, 21 years or older, 25 years or older, 30 years or older, 35 years or older, 40 years or older, 45 years or older, 50 years or older, 60 years or older, 65 years or older, 70 years or older, 75 years or older, 80 years or older, 85 years or older, 90 years or older, or 2, 3, 4, 5, 6, 7, 8, 9, 10, 12, 13, 14, 15, 16, 18, 20, 21, 24, 25, 27, 28, 30, 33, 35, 37, 39, 40, 42, 44, 45, 48, 50, 52, 55, 60, 65, 70, 75, 80, 85, 90, 95, 96, 97, 98, 99, 100, 101, 102, 103, 104, or more years old).

In addition to individuals that have been diagnosed with presbyopia, individuals considered at risk for developing presbyopia may benefit from the present disclosure, e.g., because prophylactic treatment can begin before there is any evidence and/or diagnosis of the disorder. Individuals “at risk” include, e.g., individuals having genetic or environmental factors associated with development of presbyopia, such as individuals having at least one hyperopic parent, individuals spending limited time outdoors, individuals with shorter axial lengths, and the like. Similarly, individuals in very early stages of presbyopia may benefit from prophylactic treatment.

Skilled practitioners will appreciate that a patient can be diagnosed, e.g., by a medical professional, e.g., a physician or nurse (or veterinarian, as appropriate for the patient being diagnosed), as suffering from or at risk for a condition described herein, e.g., presbyopia, using any method known in the art, e.g., by assessing a patient's medical history, performing diagnostic tests, and/or by employing imaging techniques. Various methods are known in the art to determine presbyopia and/or measure axial length.

Skilled practitioners will also appreciate that treatment need not be administered to a patient by the same individual who diagnosed the patient (or the same individual who prescribed the treatment for the patient). Treatment can be administered (and/or administration can be supervised), e.g., by the diagnosing and/or prescribing individual, and/or any other individual, including the patient her/himself (e.g., where the patient is capable of self-administration).

The active muscular mechanism of focusing of the human eye involves the change in shape and position of the lens, produced by the contraction of the ciliary muscle of the eye that increases further the refractive power of the eye. However, the lens begins to gradually lose its elasticity from childhood and its capacity to change shape and position in response to the contraction of the ciliary muscle. Around age 40, the loss of elasticity begins to affect normal eyes when they are unable to focus on near objects, e.g., less than about 40 centimeters from the eye, in a relaxed manner. From an optical standpoint, presbyopic eyes are generally affected first by this loss of elasticity due to the need to increase the refractive power to see clearly.

In the eye, the ciliary muscle controls the shape of the lens through suspended suspensory ligaments called zonules, and the ciliary muscle is controlled by the parasympathetic nervous system via acetylcholine and its muscarinic receptors. The sympathetic nervous system plays a secondary role via its alpha and beta receptors. Muscarinic agonists can increase the contraction of the ciliary muscle and hence increase the refractive power of the eye. A cholinesterase inhibitor can inhibit the acetylcholinesterase enzyme from breaking down acetylcholine, thereby increasing both the level and duration of action of the neurotransmitter acetylcholine.

Another mechanism for treating presbyopia is the effect on the dilating muscle and sphincter muscle of the iris that changes the diameter of the pupil. The iris sphincter muscle is mainly under parasympathetic control via muscarinic receptors. The iris dilating muscle is under sympathetic control, mainly alpha-1 and alpha-2 adrenergic receptors, with alpha-1 agonists producing dilation and alpha-2 agonists limiting dilation. The depth of the visual field of the eye could be increased by decreasing the diameter of the pupil. This is analogous to a photographic camera in which the depth of field increases as the diaphragm is narrowed. The use of a miotic agent (i.e., either a muscarinic agonist to activate the iris sphincter or an alpha-2 agonist to relax the dilating muscle of the iris) may constrict the pupil, thereby increasing the depth of focus of the eye.

During neurotransmission, acetylcholine is released from the nerve into the synaptic cleft and binds to acetylcholine receptors (i.e., nicotinic and muscarinic receptors) on the post-synaptic membrane, relaying the signal from the nerve. Acetylcholine esterase, also located on the post-synaptic membrane, terminates the signal transmission by hydrolyzing acetylcholine. As described herein, a cholinesterase inhibitor can potentiate the effects of endogenous acetylcholine, leading to acetylcholine accumulation, hyper-stimulation of nicotinic and muscarinic receptors, and disrupted neurotransmission. Cholinesterase inhibitors, interacting with the enzyme as their primary target, can be applied as relevant drugs or toxins. Cholinesterase inhibitors can be reversible, non-reversible, or quasi-reversible.

The ciliary muscle receives only parasympathetic fibers from the short ciliary nerves that arise from the ciliary ganglion. Parasympathetic stimulation by a cholinesterase inhibitor can cause ciliary muscle contraction. The effect of contraction is to decrease the diameter of the ring of ciliary muscle. The zonule fibers relax and the lens becomes more spherical, increasing its power to refract light for near vision.

In accordance with the present disclosure, a cholinesterase inhibitor can cause contraction of longitudinal fibers of the ciliary muscle. Acholinesterase inhibitor can be a compound that binds to and inhibits a cholinesterase enzyme from breaking down acetylcholine. This can increase both the level and duration of the neurotransmitter action. Without limiting the present disclosure to specific groups and compounds listed, the following is a list of representative cholinesterase inhibitor useful in the present disclosure: physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, and diisopropyl fluorophosphates.

The following is a summary of the properties of a few ophthalmic cholinesterase inhibitors:

TABLE 1

| | MW | Concentration | Acting | | |
|---|---|---|---|---|---|
| Compound | (Dalton) | (w/v) | Duration | | Note |
| Neostigmine | 334.4 | 3-5% | 4-6 | hours | Stable, thought to have less |
| mesylsulfate | | | | | side effects than other |
| | | | | | cholinesterase inhibitors |
| Physostigmine | 648.8 | 0.25-1% | 4-6 | hours | Can cause irritation; a |
| sulfate | | | | | discontinued eye drop |
| Demecarium | 716.6 | 0.125-0.25% | 1-2 | day | Stable; a discontinued eye |
| bromide | | | | | drop |
| Diisopropyl | 184.1 | 0.01-0.1% | — | | Not stable, can cause allergic |
| fluorophosphate | | | | | reaction |
| Echothiophate | 383.2 | 0.03-0.25% | 1-2 | day | Not stable; can cause systemic |
| iodide | | | | | side effects |

The iris has two sets of muscles: pupillary sphincter muscle that causes iris constriction and is supplied by the parasympathetic system such as muscarinic receptors, and pupillary dilator muscle that causes dilation of the iris and is supplied by the sympathetic system such as adrenergic receptors. For presbyopia treatment, when a cholinesterase inhibitor is used to modulate ciliary muscle to achieve normal accommodation, the cholinesterase inhibitor often contracts the pupillary sphincter muscle to cause pupil constriction. Since a miotic eye has a greater depth of the visual field and hence a greater depth of focus, a miotic agent in combination with cholinesterase inhibitor can improve the vision of presbyopic patient. It has surprisingly been found that a miotic agent that does not interfere with a cholinesterase inhibitor activities will act synergistically to constrict the pupil.

The compositions and methods described herein can include miotic agents. A miotic agent is a compound or composition that can contract the pupillary sphincter muscle (also called the iris sphincter muscle), or a compound or composition that can contract the ciliary muscle. In some embodiments, the miotic agent is a miotic agent that does not counteract the muscarinic activities of a muscarinic receptor antagonist. In some aspects, the miotic agent is a muscarinic agonist. In some embodiments, the miotic agent contracts the pupillary dilator muscle, and the miotic agent does not interfere with the parasympathetic system.

Exemplary types of miotic agents useful in the methods and compositions described herein include, but are not limited to, muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, a pharmaceutically acceptable salt of any of the preceding compounds, or a combination thereof.

Non-limiting examples of muscarinic receptor agonists include choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and the like, and/or a pharmaceutically acceptable salt of any of the preceding compounds, or a combination thereof.

Non-limiting examples of alpha-1 receptor antagonists include phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYM-12743, GYM-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, and the like, and/or a pharmaceutically acceptable salt of any of the preceding compounds, or a combination thereof.

Non-limiting examples of alpha-2 receptor agonists include apraclonidine, brimonidine, clonidine, mivazerol, naphazoline, oxymetazoline, tetrahydrozoline, guanfacine, guanabenz, guanoxabenz, guanethidine, xylazine, tizanidine, medetomidine, methyldopa, methylnorepinephrine, moxonidine, rilmenidine, fadolmidine, dexmedetomidine, amitraz, cannabivarin, detomidine, dihydroergotamine, dipivefrine, dopamine, ephedrine, ergotamine, esproquin, etilefrine, eEthylnorepinephrine, 6-fluoronorepinephrine, levonordefrin, lofexidine, naphazoline, 4-NEMD, (R)-3-nitrobiphenyline, norepinephrine, phenylpropanolamine, piperoxan, pseudoephedrine, rilmenidine, romifidine, talipexole, tetrahydrozoline, tolonidine, xylometazoline, and the like, and/or a pharmaceutically acceptable salt of any of the preceding compounds, or a combination thereof.

Non-limiting examples of β-adrenergic receptor antagonists include acebutolol, atenolol, betaxolol, bisoprolol, carteolol esmolol, isoproterenol, levobunolol, metoprolol, penbutolol nadolol, nebivolol, pindolol, propranolol, timolol, sotalol, and the like, and/or a pharmaceutically acceptable salt of any one or more of the preceding compounds. Non-limiting examples of nicotine receptor agonist include nicotine, varenicline, galantamine, epibatidine, lobeline, decamethonium, cytosine, nifene, dimethylphenylpiperazinium, and the like, and/or a pharmaceutically acceptable salt of any of the preceding compounds, or a combination thereof.

Non-limiting examples of adenosine receptor antagonists include 7-methylxathine, caffeine, theophylline, theobromine, and the like, and/or a pharmaceutically acceptable salt of any of the preceding compounds, or a combination thereof.

Non-limiting examples of antipsychotics include risperdal, haloperidol, thorazine, olanzapine, quetiapine, mirtazapine, chlorpromazine, prochlorperazine, alizapride, metoclopramide, midazolam, lorazepam, and the like, and/or a pharmaceutically acceptable salt of any of the preceding compounds, or a combination thereof.

Non-limiting examples of anti-emetics include ondansetron, droperidol, metoclopramide, dolasetron, granisetron, tropisetron, palonosetron, domperidone, aprepitant, casopitant, rolapitant, cyclizene, diphenhydramine, dimenhydinate, doxylamine, meclizine, promethazine, hydroxylzine, and the like, and/or a pharmaceutically acceptable salt of any one or more of the preceding compounds.

Non-limiting examples of cannabinoids include cannabis, dronebinol, nabilone, sativex, and the like, and/or a pharmaceutically acceptable salt of any of the preceding compounds, or a combination thereof.

Non-limiting examples of monoamine oxidase (MAO) inhibitors include selegiline, befloxatone moclobemide, isocarboxazid, nialamide, pheneizine, hydracarbazine, traylcypromine, bifemelane, pirlindole, toloxatone, rasagiline, linezolid, methylene blue, and the like, and/or a pharmaceutically acceptable salt of any of the preceding compounds, or a combination thereof.

Non-limiting examples of EP1 receptor agonists, EP4 receptor agonists, and FP receptor agonists include PGE2, PGE1, PGF2α, PGD2, PGE2, PGI2, TXA2, cloprostenol, flupostenol, latanoprost, tafluprost, enprostil, sulprostone, U46619, carbacyclin, and iloprost, ONO-D1-OO4, 1-hydroxy-PGE₁, rivenprost (ONO-4819), OOG-308, ONO-AE1-329, AGN205203, ONO-4819, CP-734432, AE1-329, SC-19220, SC-51089, EP4RAG, and the like, and/or a pharmaceutically acceptable salt of any of the preceding compounds, or a combination thereof.

Non-limiting examples of others miotic agents useful in the compositions and methods described herein include camptothecin (which is generally known as a cancer drug) and derivatives, ionomysin (Ca2+ channel modulator), thapsigargin (Ca2+ channel modulator), reserpine (norepinephrine depleting agents), and the like, and/or a pharmaceutically acceptable salt of any of the preceding compounds, or a combination thereof.

Analogs of the miotic agents that function as miotic agents are also embraced by the compositions and methods described herein. The ability of such analogs to exhibit miotic effects can be tested using methods known in the art.

Also provided herein are pharmaceutical composition that include a cholinesterase inhibitor and/or a miotic agent. A pharmaceutical composition is formulated to be compatible with its intended route of administration. Examples of routes of administration include ocular administration, e.g., topical ocular, injectable, ocular implant administration, or in combination with an ocular medical device such as a lens. Pharmaceutically acceptable carriers can include solvents, dispersion media, isotonic and absorption delaying agents, and the like, compatible with pharmaceutical administration. Supplementary active compounds can also be incorporated into the compositions.

The cholinesterase inhibitor and/or miotic agent described herein may be administered per se or in the form of a pharmaceutically acceptable salt. When used in a formulation, the salts should be both pharmacologically and pharmaceutically acceptable, but non-pharmaceutically acceptable salts may be conveniently used to prepare the active free compound or pharmaceutically acceptable salts thereof. A pharmaceutically acceptable salt can be any salt that retains the activity of the parent compound and does not impart any additional deleterious or untoward effects on the subject to which it is administered and in the context in which it is administered compared to the parent compound. Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring. In some embodiments, pharmaceutically acceptable salts can also include salts of active compounds which are prepared with relatively nontoxic acids or bases, depending on the particular substituent moieties found on the compounds described herein. When compounds useful in the compositions and methods described herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds useful in the compositions and methods described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66:1-19) which is incorporated by reference. In some aspects, compounds useful in the compositions and methods described herein may contain both basic and acidic functionalities that allow the compounds to be converted into either base or acid addition salts.

Some of the compounds of the described herein are known in the art for their purposes, and are known to be safe under ordinary conditions of use. Thus, in some aspects, a treatment of this disclosure can be administered by substantially conventional means, consistent with known eye treatments, while avoiding irritation, discomfort of the need for unusual application procedures.

The compositions described herein containing a cholinesterase inhibitor and/or a miotic agent and can, in some aspects, be formulated as ophthalmological compositions or formulations. In some embodiments, the cholinesterase inhibitor and miotic agent can be provided in separate formulations. In some embodiments, the cholinesterase inhibitor and miotic agent can be provided in a single formulation. Formulations useful for the compositions and methods described herein may include any formulation, in which the compositions and/or compounds described herein may be delivered to the eye. In some aspects, the cholinesterase inhibitor and/or miotic agent of the present invention are applied to the eye in a topical preparation. By a topical preparation, it is meant a preparation, which is adapted to be applied to the surface of the eye.

In some aspects of topical preparations described herein, therapeutic compounds of the preparation can contact the surface of the eye and penetrate into the deeper tissues of the eye. In some aspects, topical preparations have liquid carriers and can be aqueous solutions or suspensions, or emulsions. In some aspects, topical preparations can include a solution, a suspension, an emulsion, a gel, or a sustained release formulation, including, e.g., an implants or an ocular device such as a lens. In some aspects, the cholinesterase inhibitor and miotic agent described herein can be provided in formulations that enhance the duration of activity of the composition on neuro-effective junctions. Accordingly, such formulations may include any of the cholinesterase inhibitor or miotic agents described herein.

The compounds of the present invention may be applied in a pharmaceutically acceptable ophthalmic preparation. In some cases, such an application can have adverse effects (e.g., side effects). A pharmaceutically acceptable ophthalmic preparation can, in some aspects, produce medically desirable therapeutic effects without concurrently causing clinically significant adverse effects. Clinically significant adverse effects can refer to unacceptable side effects of the preparation, including either medically or cosmetically unacceptable effects. Non-limiting examples of possible adverse effects include eyeball and/or eyelid twitching, increased blinking times, slightly inflexible eye mobility, eye irritation, eye redness, itching, adherence of the upper eyelid and eyeball, slight weakening of upper eyelid and eyeball, headache, eye discomfort, eye ache (e.g., severe eye ache), conjunctival congestion, impaired long distance vison, elevated intraocular pressure, brow ache, excessive pupillary constriction, unacceptable light sensitivity, and the like. In some cases, adverse effects can be acceptable (e.g., and treatment can continue). Potential examples of acceptable side effects can include eyeball and/or eyelid twitching, increased blinking times, slightly inflexible eye mobility, eye irritation, eye redness, itching, adherence of the upper eyelid and eyeball, slight weakening of upper eyelid and eyeball, conjunctival congestion, and the like. In some cases, a clinical sign can be unacceptable (e.g., and either the subject or medical staff ceases treatment). Potential examples of unacceptable side effects can include reddening or irritated eyes, impaired long distance vision, elevated intraocular pressure, brow ache, headache, severe eye ache, excessive pupillary constriction, unacceptable light sensitivity, and the like; however, the determination of unacceptability can be made by the patient and/or medical staff based on the degree of the side effect. In some embodiments, exemplary pupillary constriction of less than 2.0 mm (e.g., less than 1.5 mm or 1.0 mm), in diameter is considered to be excessive.

The compounds of the present invention can be administered in therapeutically effective amounts. As described above therapeutic effective amount includes, in some aspects, an amount that slows down the progression of presbyopia with minimal pupil constriction. Compounds are typically added to the ophthalmic preparations of the disclosure in concentrations of between about 0.001% and about 10% (e.g., 0.001% to about 5%) (w/v) in the composition or formulation.

In some aspects, cholinesterase inhibitor is present in a composition or formulation described herein at a concentration of from about 0.001% to about 10%, from about 0.001% to about 4%, from about 0.001% to about 3%, from about 0.001% to about 2%, from about 0.001% to about 1.5%, from about 0.001% to about 1%, from about 0.001% to about 0.5%, from about 0.001% to about 0.2%, from about 0.001% to about 0.1%, from about 0.001% to about 0.05%, from about 0.001% to about 0.01%, from about 0.001% to about 0.005%, from about 0.01% to about 0.05%, from about 0.01% to about 0.1%, from about 0.05% to about 0.1%, from about 0.1% to about 1%, about 0.001%, about 0.002%, about 0.005%, about 0.01%, about 0.05%, about 0.083%, about 0.1%, about 0.25%, about 0.5%, about 0.75%, about 1%, about 1.1%, about 1.5%, about 1.8%, about 2%, about 3%, about 4%, or about 5%, (w/v) in the composition or formulation.

In some aspects, the miotic agent is present in a composition or formulation described herein at a concentration of from about 0.001% to about 5%, from about 0.001% to about 4%, from about 0.001% to about 3%, from about 0.001% to about 2%, from about 0.001% to about 1.5%, from about 0.001% to about 1%, from about 0.001% to about 0.5%, from about 0.001% to about 0.2%, from about 0.001% to about 0.1%, from about 0.001% to about 0.05%, from about 0.001% to about 0.01%, from about 0.001% to about 0.005%, from about 0.01% to about 0.05%, from about 0.01% to about 0.1%, from about 0.05% to about 0.1%, from about 0.1% to about 1%, about 0.001%, about 0.002%, about 0.005%, about 0.01%, about 0.05%, about 0.1%, about 0.5%, about 1%, about 1.1%, about 1.5%, about 1.8%, about 2%, about 3%, about 4%, or about 5%, (w/v) in the composition or formulation.

In some aspects, the compounds of the present disclosure are preferably administered topically and delivered in a medically acceptable, substantially sterile, non-irritating composition or formulation (e.g., an ophthalmic preparation). The compositions or formulations (e.g., ophthalmic preparations) can contain pharmaceutically acceptable concentrations of salts, buffering agents, preservatives, viscosity modifiers, osmotic agent and delivery enhancing agents. Exemplary non-limiting salts which can be used include sodium chloride, zinc sulfate, and potassium chloride. A salt can be used in any appropriate concentration. For example, salt (e.g., sodium chloride) can be used in a concentration of about 0.1% to about 0.5% (w/v) (e.g., about 0.2% to about 0.4%, about 0.1% to about 0.3%, about 0.3% to about 0.5%, about 0.1%, about 0.2%, about 0.3%, about 0.4%, or about 0.5% (w/v)). Exemplary non-limiting buffers which can be used include boric acid and citric acid. A buffer can be used in any appropriate concentration. For example, a buffer (e.g., boric acid) can be used in a concentration of about 0.5% to about 2.0% (w/v) (e.g., about 0.5% to about 1.0%, about 1.0% to about 1.5%, about 1.5% to about 2.0%, about 1% to about 2%, about 0.5% to about 1.5%, about 0.75% to about 1.25%, about 0.9% to about 1.1%, about 0.5%, about 0.75%, about 1.0%, about 1.25%, about 1.5%, about 1.75%, or about 2.0% (w/v)). Exemplary non-limiting preservatives which can be used include benzalkonium chloride and edetate disodium. A preservative can be used in any appropriate concentration. For example, a preservative can be used in a concentration of about 0.005% to about 0.5% (w/v) (e.g., about 0.005% to about 0.01%, about 0.01% to about 0.05%, about 0.05% to about 0.1%, about 0.1% to about 0.5%, about 0.005% to about 0.05%, about 0.05% to about 0.5%, about 0.01% to about 0.1%, about 0.005%, about 0.01%, about 0.05%, about 0.1%, or about 0.5% (w/v)). For example, benzalkonium chloride can be used in a concentration of about 0.005% to about 0.05% (e.g., about 0.005% to about 0.007%, about 0.007% to about 0.009%, about 0.011%, about 0.01% to about 0.02%, about 0.02% to about 0.03%, about 0.03% to about 0.04%, about 0.04% to about 0.05%, about 0.005% to about 0.02%, about 0.005%, about 0.007%, about 0.009%, about 0.01%, about 0.02%, about 0.03%, about 0.04%, or about 0.05% (w/v)). For example, edetate disodium can be used in a concentration of about 0.05% to about 5% (w/v) (e.g., about 0.05% to about 0.07%, about 0.07% to about 0.09%, about 0.09% to about 0.11%, about 0.1% to about 0.2%, about 0.2% to about 0.3%, about 0.3% to about 0.4%, about 0.4% to about 0.5%, about 0.05% to about 0.2%, about 0.05%, about 0.07%, about 0.09%, about 0.1%, about 0.2%, about 0.3%, about 0.4%, or about 0.5% (w/v)). Exemplary non-limiting viscosity modifiers which can be used include methyl cellulose, carboxymethyl cellulose, hydroxypropylmethyl cellulose), glycerol, and polyethylene glycol. A viscosity modifier can be used in any appropriate concentration. Exemplary non-limiting osmotic agents which can be used include mannitol and sorbitol. An osmotic agent can be used in any appropriate concentration Exemplary non-limiting delivery enhancing agents that facilitate the delivery of the therapeutic compound(s) of the disclosure into the aqueous humor, include substances which increase corneal permeability, such as surfactants (e.g., an anionic surfactant, a nonionic surfactant, or a combination thereof), solubilizers, wetting agents, liposomes, DMSO, and the like, each of which can be used in any appropriate concentration. Exemplary solubilizers include cyclodextrins (e.g., a gamma cyclodextrin, a beta-cyclodextrin (e.g., sulfobutylether β-cyclodextrin or 2-hydroxypropyl beta-cyclodextrin)). Exemplary anionic surfactants include sodium lauryl sulfate and sodium ester lauryl sulfate. Exemplary nonionic surfactants include a poloxamer, a polysorbate, sorbitan monolaurate, sorbitan monopalmitate, sorbitan monostearate, sorbitan monooleate, polyoxyl stearate (e.g., polyolyl 40 stearate), a polyoxyl alkyl, and derivatives thereof.

In some embodiments, composition or formulation (e.g., ophthalmic preparation) can include a cholinesterase inhibitor (e.g., about 0.01% to about 2.0% (w/v) (e.g., about 0.01% to about 0.05%, about 0.01% to about 0.1%, about 0.05% to about 0.1%, about 0.1% to about 0.5%, about 0.5% to about 1.0%, about 1.0% to about 2.0%, about 1.0% to about 1.5%, about 1.5% to about 2.0%, about 0.05%, about 0.083%, about 0.1%, about 0.25%, about 0.75%, about 1.0%, or about 1.5% (w/v))), a miotic agent (e.g., about 0.01% to about 5.0% (w/v) (e.g., about 0.01% to about 0.05%, about 0.01% to about 0.1%, about 0.05% to about 0.1%, about 0.1% to about 0.5%, about 0.5% to about 1.0%, about 1.0% to about 2.0%, about 2.0 to about 5.0%, about 1.0% to about 1.5%, about 1.5% to about 2.0%, about 0.05%, about 0.083%, about 0.1%, about 0.25%, about 0.75%, about 1.0%, or about 1.5% (w/v))), about 0.0.01, or a combination thereof and about 0.2% to about 0.4% (e.g., about 0.3%) (w/v) of a salt (e.g., sodium chloride), about 0.8% to about 1.2% (e.g., about 1.0%) (w/v) of a buffer (e.g., boric acid), about 0.08% to about 0.15% (e.g., about 0.1% or about 0.11%) (w/v) of a preservative (e.g., edetate disodium and/or benzalkonium chloride (e.g., about 0.08% to about 0.12% (e.g., about 0.1%) (w/v) of edetate disodium and about 0.008% to about 0.012% (e.g., about 0.01%) (w/v) of benzalkonium chloride)) at a pH of about 6.5 to about 7.5 (e.g., about 6.8 to about 7.2 or about 7.0).

An antioxidant is usually used to stabilize a composition or formulation (e.g., ophthalmic preparation) (e.g., a solution) against oxidation by free radicals and oxygen in the air. Common antioxidants include ascorbic acid, glutathione, acetylcysteine, monothioglycerol, metabisulfite salts, bisulfite salts, tris(2-carboxyethyl)phosphine (TCEP), dithiothreitol (DTT), dithiobutylamine (DTBA), tris(3-hydroxypropyl)phosphine (THPP). An antioxidant can be used in any appropriate concentration.

A chelating agent can remove the metal ions from a composition or formulation (e.g., ophthalmic preparation) (e.g., a solution), hence reduce the potential of metal ion catalyzed oxidation reaction. The commonly used pharmaceutical chelating ingredients include, but not limited to, the following: ethylenediaminetetraacetic acid (EDTA), calcium disodium EDTA, disodium EDTA (also called edetate disodium), pentetic acid (DTPA), N-(2-Hydroxyethyl)ethylenediaminetriacetic acid (HEDTA), nitrilotriacetic acid (NTA), methylglycinediacetic acid (MGDA), ethylenediaminedisuccinicacid (EDDS), and iminodisuccinic acid (IDS). A chelating can be used in any appropriate concentration.

A wetting agent is a substance which facilitates corneal penetration by mildly disrupting the outer corneal surface. In some aspects, the wetting agent is benzalkonium chloride. Other non-limiting examples of wetting agents include sorbitan esters, and polyoxyethylene ethers. It should be understood that although specific formulations have been defined, many variations are possible. The compositions or formulations (e.g., ophthalmic preparations) useful in the eye are nonirritating and non-damaging to the eye in the preferred form, and are effective to provide the results desired. A wetting agent can be used in any appropriate concentration.

In some aspects, a composition or formulation (e.g., an ophthalmic preparation) can be applied in a liquid carrier. In some aspects, the carrier is an aqueous carrier. In some aspects, the carrier is water. In some aspects, quick dissolving forms of the medicaments may be administered in powder form or rubbed into the eye from applicators of various types. Spraying of the eye, eye drops, and other methods of application can be used.

In some aspects, the preparations are packaged as sterile solutions in dropper bottles, as are standard in ophthalmic preparations. Other containers, including eye cups, can also be used. The preparation can, in some aspects, be packaged with instructions for using the preparation in treating presbyopia, in some aspects, directing the use of preparation to administer 1 to 2 drops of the solution to each eye.

In some aspects, a composition as described herein can be administered in a pharmaceutically acceptable formulation (e.g., an ophthalmic preparation), such as topically by application of the formulation to the eye in a non-irritating sterile solution or suspension. In that regard, the formulation is preferably at a pH compatible with the eye (e.g., about 6.5 to about 7.5).

Dosage levels will vary greatly depending upon the individual to be treated, the progression of the disorder, and the specific medicament(s) used. One of ordinary skill in the art, such as a health care provider, can determine proper dosing without undue experimentation and according to standard procedures in the art. Exemplary dosage amounts useful in some embodiments of the methods described herein include 1-2 drops per application. In some embodiments, drop sizes range from about 30 μL to about 80 pt. In some embodiments, exemplary dosage amounts can range from about 30 μL to about 480 μL per application. Exemplary dosage regimens useful in some aspects of the methods described herein include 1 application per day, two applications per day, three applications per day, four applications per day, five applications per day, six applications per day, one application every other day, on application per week, two applications per week, or three applications per week.

In some embodiments, compositions or formulations described herein (e.g., compositions or formulations having a concentration of from about 0.001% to about 2% (w/v) of a cholinesterase antagonist, and/or having a concentration of from about 0.001% to about 5% (w/v) of the miotic agent) can be administered in an application having an amount of from about 1 μL to about 480 μL per application. In some embodiments, application amounts can range from about 10 μL to about 400 μL, from about 20 μL to about 300 μL, from about 20 μL to about 250 μL, from about 20 μL to about 200 μL, from about 20 μL to about 150 μL, from about 20 μL to about 100 μL, from about 25 μL to about 90 μL, from about 25 μL to about 85 μL, from about 30 μL to about 80 μL, from about 25 μL to about 50 μL, from about 25 μL to about 45 μL, from about 25 μL to about 40 μL, from about 30 μL to about 40 μL, from about 55 μL to about 90 μL, from about 60 μL to about 85 μL, or from about 60 μL to about 80 μL per application.

In some embodiments, compositions or formulations described herein (e.g., compositions or formulations including the cholinesterase antagonist, the miotic agent, or a combination thereof) can be administered during a treatment period. In some aspects, exemplary treatment periods include 1 day, up to about 5 days, up to about 10 days, up to about 30 days, up to about 1 week, up to about 2 weeks, up to about 3 weeks, up to about 4 weeks, up to about 5 weeks, up to about 1 month, up to about 2 months, up to about 3 months, up to about 4 months, up to about 5 months, up to about 6 months, up to about 7 months, up to about 8 months, up to about 9 months, up to about 10 months, up to about 11 months, up to about 1 year, up to about 2 years, up to about 3 years, up to about 4 years, up to about 5 years, or up to about 10 years, from about 1 day to about 10 years, from about 1 month to about 10 years, from about 2 months to about 10 years, from about 3 months to about 10 years, from about 4 months to about 10 years, from about 5 months to about 10 years, from about 6 months to about 10 years, from about 6 months to about 9 years, from about 6 months to about 8 years, from about 6 months to about 7 years, from about 6 months to about 6 years, from about 6 months to about 5 years, from about 1 day to about 60 months, from about 6 months to about 4 years, from about 6 months to about 3 years, from about 6 months to about 2 years, from about 6 months to about 1 year, and the like. In some aspects of the methods described herein, treatment regimens may be periodically stopped and restarted according to the subject's needs.

In some aspects, compositions or formulations described herein (e.g., compositions or formulations including the cholinesterase antagonist, the miotic agent, or a combination thereof) can be administered from 1 to 6 times per day, from 1 to 5 times per day, from 1 to 4 times per day, from 1 to 3 times per day, or from 1 to 2 times per day during the treatment period. In some embodiments, compositions or formulations described herein (e.g., compositions or formulations including the cholinesterase antagonist, the miotic agent, or a combination thereof) can be administered to a subject within, e.g., 2 hours, 1 hour, 45 minutes, 30 minutes, 15 minutes, 10 minutes, or 5 minutes prior to a period of sleep for the subject.

Provided herein are methods comprising administering to an eye of a subject, during a treatment period, a cholinesterase inhibitor and a pharmaceutically acceptable carrier. Also provided herein are methods comprising administering to an eye of a subject, during a treatment period, a miotic agent and a pharmaceutically acceptable carrier. In some aspects, provided herein are methods comprising administering to an eye of a subject, during a treatment period, a cholinesterase inhibitor, a miotic agent, and a pharmaceutically acceptable carrier. In some embodiments, the methods are methods of treating presbyopia in a subject and comprise administering to an affected eye of the subject a therapeutically effective amount of the cholinesterase inhibitor, miotic agent, or a combination thereof, and a pharmaceutically acceptable carrier. Provided herein are methods for inhibiting and/or slowing axial shortening of an affected eye of a subject, while minimizing side effects such as, e.g., pupil constriction and/or light sensitivity in the subject. In some aspects, the methods can be useful in treating, e.g., presbyopia in an effected eye. Also provided herein are methods for treating presbyopia in an affected eye of a subject. In some aspects, the compositions and methods can be useful preventing, inhibiting, slowing, or regressing the progression of presbyopia in an eye while minimizing side effects such as, e.g., pupil constriction and/or light sensitivity in the subject. Exemplary methods include administering to a subject in need of treatment therapeutically effective amounts of any of the compositions or formulations described herein comprising a cholinesterase antagonist, a miotic agent, or a combination thereof. Some exemplary methods include administering to the subject a therapeutically effective amount of a cholinesterase antagonist. Some exemplary methods include administering to the subject a therapeutically effective amount of a miotic agent. Other exemplary methods include administering to the subject therapeutically effective amounts of each of a cholinesterase antagonist and a miotic agent. In some aspects of the methods described herein, the cholinesterase antagonist and the miotic agent can be administered concurrently. In some aspects of the methods described herein, the cholinesterase antagonist and the miotic agent can be administered subsequently. In some aspects of the methods described herein, the cholinesterase antagonist can be administered prior to administering the miotic agent. In some aspects of the methods described herein, the cholinesterase antagonist can be administered after the miotic agent. In various aspects of the methods described herein, the methods can treat disease in the subject. In some aspects of the methods described herein, the cholinesterase antagonist, the miotic agent, or the combination thereof is administered to an affected eye of the subject.

In some aspects of any of the methods described herein, the subject has or is at risk for developing presbyopia in at least one eye. For example, in some aspects, methods described herein can also be useful for treatment of presbyopia. As another example, in some aspects, methods described herein can also be useful for prophylactic treatment of presbyopia. As used herein, treating includes “prophylactic treatment”, which means reducing the incidence of or preventing (or reducing the risk of) a sign or symptom of a disease (e.g., presbyopia) in a subject at risk of developing a disease (e.g., presbyopia). The method described herein is suited particularly for subjects who are otherwise free of indications for ophthalmic treatments utilizing a cholinesterase antagonist and/or a miotic agent. In some aspects of the methods described herein, the cholinesterase antagonist, miotic agent, or combination thereof is administered in an amount sufficient to inhibit, slow, or prevent progression of presbyopia in the eye. In some aspects of the methods described herein, the cholinesterase antagonist, miotic agent, or combination thereof is administered in an amount sufficient to inhibit or slow shrinkage in the axial length of the eye. In some aspects of the methods described herein, the cholinesterase inhibitor, miotic agent, or combination thereof is administered in an amount sufficient to maintain pupillary dilation of e.g., at least about 2.0 mm, at least about 2.1 mm, at least about 2.2 mm, at least about 2.3 mm, at least about 2.4 mm, at least about 2.5 mm, at least about 2.6 mm, at least about 2.7 mm, at least about 2.8 mm, at least about 2.9 mm, at least about 3.0 mm, at least about 3.2 mm, at least about 3.4 mm, at least about 3.6 mm, at least about 3.8 mm, at least about 4.0 mm, at least about 4.5 mm, or at least about 5.0 mm in diameter. In some aspects of the methods described herein, the cholinesterase inhibitor, miotic agent, or combination thereof is administered in an amount sufficient to achieve a Log MAR visual acuity (e.g., using a distance corrected near visual acuity (DCNVA) at 40 cm) of less than about 0.3 (e.g., less than about 0.28, 0.26, 0.25, 0.24, 0.22, 0.20, 0.18, 0.16, 0.15, 0.14, 0.12, or 0.10), for example, in an affected eye of a subject.

In some aspects, methods described herein can include treating the eye with an ocular device during the treatment period. In some aspects, the ocular device can be a lens, such as a contact lens, an implanted lens, or a lens associated with external devices such as glasses. In some aspects the ocular device can be a corrective lens. In some aspects, a contact lenses or implantable lenses may contain or be treated with any of the compositions described herein to provide a route of administration for the compositions.

In some aspects, the methods described herein can result in a decreased need (e.g., in terms of frequency of use, prescription strength, or need for bifocals) or removal of need for an ocular device (e.g., glasses or contact lenses) during the treatment period. For example, a subject treated with a composition or formulation described herein may have a decreased need of using an ocular device (e.g., as compared to such a need before treatment) during the treatment period. As another example, a subject treated with a composition or formulation described herein may not need an ocular device (e.g., as compared to such a need before treatment) during the treatment period.

In some aspects of the methods described herein, parasympathetic/cholinergic/ciliary contraction in the subject's eye are increased in order to allow normal accommodation for a presbyopic eye. Without wishing to be bound by theory, it is believed this action of the ciliary muscle under parasympathetic innervation provides for zonules relaxation, which can allow the lens to assume a more spherical shape.

In some aspects, a method can include administering to a presbyoptic subject an effective amount of a cholinesterase antagonist in combination with a miotic agent. The selection of a cholinesterase antagonist in combination with a miotic agent can slow down the progression of presbyopia and minimize the effects on pupillary muscle to reduce the potential adverse effects.

Compositions described herein can include additional therapeutic agents. Methods described herein can also include administration of additional therapeutic agents. The term “therapeutic agent” refers to a therapeutic treatment that involves administering to a subject a therapeutic agent that is known to be useful in the treatment of a disease, e.g., presbyopia, or known to be useful in providing a therapeutic benefit to one or more ocular diseases, discomforts, or symptoms, whether associated with a particular ocular disease or not. In some aspects, a therapeutic agent can increase comfort of a subject eyes. For example, a therapeutic agent can include a substance known to alleviate dry eyes in a subject.

Non-limiting examples of therapeutic agents include antibiotics, steroids, artificial tears, intra-ocular pressure (IOP) lowering agents, immunosuppressants, dry eye-treating agents, and the like.

Also provided herein are kits that include a composition or formulation having a cholinesterase antagonist, and a composition or formulation having a miotic agent. Also provided herein are kits that include a composition or formulation having a cholinesterase antagonist and a miotic agent. In some instances, the kits can include instructions for performing any of the methods described herein. In some aspects, the kits can include at least one dose of any of the pharmaceutical compositions described herein. The kits described herein are not so limited; other variations will be apparent to one of ordinary skill in the art.

Embodiment 1 is a method, the method comprising administering to an eye of a subject, during a treatment period, a cholinesterase inhibitor.
Embodiment 2 is the method of embodiment 1, wherein the method further comprises administering to the eye of the subject, during the treatment period, a miotic agent.
Embodiment 3 is a method, the method comprising administering to an eye of a subject, during a treatment period, a miotic agent.
Embodiment 4 is the method of embodiment 3, wherein the method further comprises administering to the eye of the subject, during the treatment period, a cholinesterase inhibitor.
Embodiment 5 is the method of any one of claim 1, 2, or 4, wherein the cholinesterase inhibitor is selected from the group consisting of physostigmine, neostigmine, pyridostigmine, ambenonium, demecarium bromide, rivastigmine, galantamine, caffeine, to rosmarinic acid, alpha-pinene, donepezil, tacrine, edrophonium, huperzine A, ladostigil, ungeremine, lactucopicrin, echothiophate, diisopropyl fluorophosphates, pharmaceutically acceptable salts thereof, and combinations thereof.
Embodiment 6 is the method of embodiment 5, wherein the cholinesterase inhibitor is selected from the group consisting of physostigmine, neostigmine, demecarium bromide, diisopropyl flurophosphate, pharmaceutically acceptable salts thereof, and combinations thereof.
Embodiment 7 is the method of any one of claim 1, 2, or 4, wherein the cholinesterase inhibitor comprises physostigmine or a pharmaceutically acceptable salt thereof.
Embodiment 8 is the method of any one of claim 1, 2, or 4, wherein the cholinesterase inhibitor comprises neostigmine or a pharmaceutically acceptable salt thereof.
Embodiment 9 is the method of any one of claim 1, 2, or 4, wherein the cholinesterase inhibitor comprises demecarium bromide.
Embodiment 10 is the method of any one of claim 1, 2, or 4, wherein the cholinesterase inhibitor comprises diisopropyl flurophosphate.
Embodiment 11 is the method of any one of claim 1, 2, or 4-10, wherein the cholinesterase inhibitor is administered at a concentration of about 0.01% to about 2.0% (w/v).
Embodiment 12 is the method of embodiment 11, wherein the cholinesterase inhibitor is administered at a concentration of about 0.05% to about 1.50% (w/v).
Embodiment 13 is the method of embodiment 11, wherein the cholinesterase inhibitor is administered at a concentration of about 0.05% (w/v).
Embodiment 14 is the method of embodiment 11, wherein the cholinesterase inhibitor is administered at a concentration of about 0.25% (w/v).
Embodiment 15 is the method of embodiment 11, wherein the cholinesterase inhibitor is administered at a concentration of about 0.75% (w/v).
Embodiment 16 is the method of embodiment 11, wherein the cholinesterase inhibitor is administered at a concentration of about 0.083% (w/v).
Embodiment 17 is the method of embodiment 11, wherein the cholinesterase inhibitor is to administered at a concentration of about 1.0% (w/v).
Embodiment 18 is the method of embodiment 11, wherein the cholinesterase inhibitor is administered at a concentration of about 1.5% (w/v).
Embodiment 19 is the method of any one of claims 2-18, wherein the miotic agent is selected from the group consisting of muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof.
Embodiment 20 is the method of any one of claims 2-19, wherein the miotic agent comprises a muscarinic receptor agonist.
Embodiment 21 is the method of embodiment 20, wherein the muscarinic receptor agonist is selected from the group consisting of choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and combinations thereof.
Embodiment 22 is the method of any one of claims 2-21, wherein the miotic agent comprises an alpha-1 adrenergic receptor antagonist.
Embodiment 23 is the method of embodiment 22, wherein the alpha-1 adrenergic receptor antagonist is selected from the group consisting of phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYKI-12743, GYKI-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof.
Embodiment 24 is the method of any one of claims 2-23, wherein the miotic agent comprises an alpha-2 adrenergic receptor agonist.
Embodiment 25 is the method of embodiment 24, wherein the alpha-2 adrenergic receptor agonist is selected from the group consisting ofapraclonidine, brimonidine, clonidine, mivazerol, naphazoline, oxymetazoline, tetrahydrozoline, guanfacine, guanabenz, guanoxabenz, guanethidine, xylazine, tizanidine, medetomidine, methyldopa, methylnorepinephrine, moxonidine, rilmenidine, fadolmidine, dexmedetomidine, amitraz, cannabivarin, detomidine, dihydroergotamine, dipivefrine, dopamine, ephedrine, ergotamine, esproquin, etilefrine, eEthylnorepinephrine, 6-fluoronorepinephrine, levonordefrin, lofexidine, naphazoline, 4-NEMD, (R)-3-nitrobiphenyline, norepinephrine, phenylpropanolamine, piperoxan, pseudoephedrine, rilmenidine, romifidine, talipexole, tetrahydrozoline, tolonidine, xylometazoline, a pharmaceutically acceptable salt thereof, and combinations thereof.
Embodiment 26 is the method of any one of claims 2-25, wherein the miotic agent is administered at a concentration of about 0.01% to about 2.0% (w/v).
Embodiment 27 is the method of embodiment 26, wherein the miotic agent is administered at a concentration of about 0.05% to about 1.50% (w/v).
Embodiment 28 is the method of any one of claims 1-27, wherein the eye of the subject is presbyoptic or is at risk for developing presbyopia.
Embodiment 29 is the method of any one of claims 1-28, wherein the amount of the cholinesterase inhibitor, miotic agent, or combination thereof is sufficient to inhibit, slow, or prevent progression of presbyopia in the eye.
Embodiment 30 is the method of any one of claims 1-29, wherein the amount of the cholinesterase inhibitor, miotic agent, or combination thereof is sufficient to maintain a pupillary dilation of at least about 2.0 mm.
Embodiment 31 is the method of embodiment 30, wherein the amount of the cholinesterase inhibitor, miotic agent, or combination thereof is sufficient to maintain a pupillary dilation of at least about 2.5 mm.
Embodiment 32 is the method of embodiment 30, wherein the amount of the cholinesterase inhibitor, miotic agent, or combination thereof is sufficient to maintain a pupillary dilation of at least about 3.0 mm.
Embodiment 33 is the method of embodiment 30, wherein the amount of the cholinesterase inhibitor, miotic agent, or combination thereof is sufficient to maintain a pupillary dilation of at least about 3.5 mm.
Embodiment 34 is the method of any one of claims 1-33, wherein the amount of the cholinesterase inhibitor, miotic agent, or combination thereof is sufficient to achieve a Log MAR visual acuity of less than about 0.3.
Embodiment 35 is the method of embodiment 34, wherein the amount of the cholinesterase inhibitor, miotic agent, or combination thereof is sufficient to achieve a Log MAR visual acuity of less than about 0.28.
Embodiment 36 is the method of embodiment 34, wherein the amount of the cholinesterase inhibitor, miotic agent, or combination thereof is sufficient to achieve a Log MAR visual acuity of less than about 0.25.
Embodiment 37 is the method of embodiment 34, wherein the amount of the cholinesterase inhibitor, miotic agent, or combination thereof is sufficient to achieve a Log MAR visual acuity of less than about 0.20.
Embodiment 38 is the method of any one of claims 34-37, wherein the Log MAR visual acuity is assessed using a distance corrected near visual acuity (DCNVA) at 40 cm.
Embodiment 39 is the method of embodiment 2 or embodiment 4, wherein the cholinesterase inhibitor and miotic agent are administered concurrently.
Embodiment 40 is the method of embodiment 40, wherein the cholinesterase inhibitor and miotic agent are administered concurrently as separate agents.
Embodiment 41 is the method of embodiment 40, wherein the cholinesterase inhibitor and miotic agent are administered concurrently as a single formulation.
Embodiment 42 is the method of embodiment 2 or embodiment 4, wherein the cholinesterase inhibitor and miotic agent are administered sequentially.
Embodiment 43 is the method of embodiment 42, wherein the cholinesterase inhibitor is administered before the miotic agent.
Embodiment 44 is the method of embodiment 42, wherein the cholinesterase inhibitor is administered after the miotic agent.
Embodiment 45 is the method of any one of claims 1-44, wherein the treatment period is from about 1 day to about 60 months.
Embodiment 46 is the method of any one of claim 1, 2, or 4-45, wherein the cholinesterase inhibitor is administered from 1-6 times per day during the treatment period.
Embodiment 47 is the method of any one of claim 1, 2, or 4-46, wherein the cholinesterase inhibitor is administered in an application in an amount of about 30 μL to about 80 μL per application.
Embodiment 48 is the method of any one of claims 2-47, wherein the miotic agent is administered from 1-6 times per day during the treatment period.
Embodiment 49 is the method of any one of claims 2-48, wherein the miotic agent is administered in an application in an amount of about 30 μL to about 80 μL per application.
Embodiment 50 is the method of any one of claims 1-49, further comprising treating the eye with an ocular device during at least a portion of the treatment period.
Embodiment 51 is the method of embodiment 50, wherein the ocular device is a lens or an implant.
Embodiment 52 is the method of embodiment 50, wherein the ocular device is a corrective lens.
Embodiment 53 is the method of any one of claims 1-52, further comprising correcting vision to in the eye with a corrective lens during the treatment period.
Embodiment 54 is the method of embodiment 53, wherein the need of the subject for correcting vision in the eye with a corrective lens is decreased during the treatment period.
Embodiment 55 is the method of embodiment 54, wherein the need of the subject for correcting vision in the eye with a corrective lens is eliminated during the treatment period.
Embodiment 56 is a composition comprising:

a. a cholinesterase inhibitor; and

b. a pharmaceutically acceptable carrier.

Embodiment 57 is the composition of embodiment 56, further comprising a miotic agent.
Embodiment 58 is a composition comprising:

a. a miotic agent; and

b. a pharmaceutically acceptable carrier.

Embodiment 59 is the composition of embodiment 58, further comprising a cholinesterase inhibitor.
Embodiment 60 is the composition of any one of claim 56, 57, or 59, wherein the cholinesterase inhibitor is selected from the group consisting of physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphates, pharmaceutically acceptable salts thereof, and combinations thereof.
Embodiment 61 is the composition of embodiment 60, wherein the cholinesterase inhibitor is selected from the group consisting of physostigmine, neostigmine, demecarium bromide, diisopropyl flurophosphate, pharmaceutically acceptable salts thereof, and combinations thereof.
Embodiment 62 is the composition any one of claim 56, 57, or 59, wherein the cholinesterase inhibitor comprises physostigmine or a pharmaceutically acceptable salt thereof.
Embodiment 63 is the composition any one of claim 56, 57, or 59 wherein the cholinesterase inhibitor comprises neostigmine or a pharmaceutically acceptable salt thereof.
Embodiment 64 is the composition any one of claim 56, 57, or 59, wherein the cholinesterase inhibitor comprises demecarium bromide.
Embodiment 65 is the composition any one of claim 56, 57, or 59, wherein the cholinesterase inhibitor comprises diisopropyl flurophosphate.
Embodiment 66 is the composition any one of claim 56, 57, or 59-65, wherein the cholinesterase inhibitor is present at a concentration of about 0.01% to about 2.0% (w/v).
Embodiment 67 is the composition of embodiment 66, wherein the cholinesterase inhibitor is present at a concentration of about 0.05% to about 1.50% (w/v).
Embodiment 68 is the composition of embodiment 66, wherein the cholinesterase inhibitor is present at a concentration of about 0.05% (w/v).
Embodiment 69 is the composition of embodiment 66, wherein the cholinesterase inhibitor is present at a concentration of about 0.25% (w/v).
Embodiment 70 is the composition of embodiment 66, wherein the cholinesterase inhibitor is present at a concentration of about 0.75% (w/v).
Embodiment 71 is the composition of embodiment 66, wherein the cholinesterase inhibitor is present at a concentration of about 0.083% (w/v).
Embodiment 72 is the composition of embodiment 66, wherein the cholinesterase inhibitor is present at a concentration of about 1.0% (w/v).
Embodiment 73 is the composition of embodiment 66, wherein the cholinesterase inhibitor is present at a concentration of about 1.5% (w/v).
Embodiment 74 is the composition of any one of claims 57-73, wherein the miotic agent is selected from the group consisting of muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof.
Embodiment 75 is the composition of any one of claims 57-73, wherein the miotic agent comprises a muscarinic receptor agonist.
Embodiment 76 is the composition of embodiment 75, wherein the muscarinic receptor agonist is selected from the group consisting of choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and combinations thereof.
Embodiment 77 is the composition of any one of claims 57-73, wherein the miotic agent comprises an alpha-1 adrenergic receptor antagonist.
Embodiment 78 is the composition of embodiment 77, wherein the alpha-1 adrenergic receptor antagonist is selected from the group consisting of phenoxybenzamine, phentolamine, tolazoline, trazodone, alfuzosin, dapiprazole, thymoxamine, doxazocin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYM-12743, GYM-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof.
Embodiment 79 is the composition of any one of claims 57-78, wherein the miotic agent comprises an alpha-2 adrenergic receptor agonist.
Embodiment 80 is the composition of embodiment 79, wherein the alpha-2 adrenergic receptor agonist is selected from the group consisting of apraclonidine, brimonidine, clonidine, mivazerol, naphazoline, oxymetazoline, tetrahydrozoline, guanfacine, guanabenz, guanoxabenz, guanethidine, xylazine, tizanidine, medetomidine, methyldopa, methylnorepinephrine, moxonidine, rilmenidine, fadolmidine, dexmedetomidine, amitraz, cannabivarin, detomidine, dihydroergotamine, dipivefrine, dopamine, ephedrine, ergotamine, esproquin, etilefrine, eEthylnorepinephrine, 6-fluoronorepinephrine, levonordefrin, lofexidine, naphazoline, 4-NEMD, (R)-3-nitrobiphenyline, norepinephrine, phenylpropanolamine, piperoxan, pseudoephedrine, rilmenidine, romifidine, talipexole, tetrahydrozoline, tolonidine, xylometazoline, a pharmaceutically acceptable salt thereof, and combinations thereof.
Embodiment 81 is the composition of any one of claims 57-80, wherein the miotic agent is present at a concentration of about 0.01% to about 2.0% (w/v).
Embodiment 82 is the composition of embodiment 81, wherein the miotic agent is present at a concentration of about 0.05% to about 1.50% (w/v).
Embodiment 83 is the composition of any one of claims 56-82, further comprising a salt.
Embodiment 84 is the composition of embodiment 83, wherein the salt is present in a concentration of about 0.2% to about 0.4% (w/v).
Embodiment 85 is the composition of any one of claims 83-84, wherein the salt comprises sodium chloride, zinc sulfate, potassium chloride, or a combination thereof.
Embodiment 86 is the composition of any one of claims 56-85, further comprising a buffer.
Embodiment 87 is the composition of embodiment 86, wherein the buffer is present in an amount of about 0.8% to about 1.2% (w/v).
Embodiment 88 is the composition of any one of claims 86-87, wherein the buffer comprises boric acid, citric acid, or a combination thereof.
Embodiment 89 is the composition of any one of claims 56-88, further comprising a preservative.
Embodiment 90 is the composition of embodiment 89, wherein the preservative is present in an amount of about 0.08% to about 0.15% (w/v).
Embodiment 91 is the composition of any one of claims 89-90, wherein the preservative comprises benzalkonium chloride, edetate disodium, or a combination thereof.
Embodiment 92 is the composition of any one of claims 56-91, wherein the pH of the composition is about 6.5 to about 7.5.
Embodiment 93 is the composition of any one of claims 56-92, further comprising a viscosity modifier.
Embodiment 94 is the composition of embodiment 93, wherein the viscosity modifier comprises methyl cellulose, carboxymethyl cellulose, hydroxypropylmethyl cellulose, glycerol, polyethylene glycol, or combinations thereof.
Embodiment 95 is the composition of any one of claims 56-94, further comprising an osmotic agent.
Embodiment 96 is the composition of embodiment 95, wherein the osmotic agent comprises mannitol, sorbitol, or a combination thereof.
Embodiment 97 is the composition of any one of claims 56-96, further comprising a surfactant, a solubilizer, a wetting agent, liposomes, DMSO, or a combination thereof.
Embodiment 98 is the composition of embodiment 97, wherein the solubilizer comprises a cyclodextrin.
Embodiment 99 is the composition of any one of claims 97-98, wherein the surfactant comprises an anionic surfactant, a nonionic surfactant, or a combination thereof.
Embodiment 100 is the composition of any one of claims 56-99, wherein the composition is a topical preparation.
Embodiment 101 is the composition of embodiment 100, wherein the composition is a solution, a suspension, an emulsion, a gel, or a sustained release formulation.
Embodiment 102 is the composition of any one of claims 56-101, wherein the composition is an ophthalmic preparation.
Embodiment 103 is a method of treating presbyopia, the method comprising administering to an affected eye of a subject in need of such treatment a therapeutically effective amount of a composition of any one of claims 56-102.
Embodiment 104 is Use of a composition of any one of claims 56-102 manufacture of a medicament for the treatment of presbyopia.
Embodiment 105 is a kit comprising a composition of any one of claims 56-102.
Embodiment 106 is An ophthalmological composition to treat presbyopia comprising of a cholinesterase inhibitor, which is a reversible, non-reversible, or quasi-reversible cholinesterase inhibitor, and can be used alone or in combination with other pharmaceutical agent.
Embodiment 107 is the ophthalmological composition according to embodiment 106, wherein the cholinesterase inhibitor is selected from the group consisting of physostigmine, neostigmine, caffeine, donepezil, echothiophate, diisopropyl fluorophosphates, demecarium.
Embodiment 108 is An ophthalmological composition to treat presbyopia comprising of a miotic agent, which is used alone or in combination with other pharmaceutical agent.
Embodiment 109 is the ophthalmological composition according to embodiment 108, wherein the miotic agent is alpha-1 adrenergic receptor antagonist, alpha-2 adrenergic receptor agonist, beta-adrenergic receptor antagonist, nicotine receptor agonist, antipsychotics, anti-emetics, cannabinoid, MAO inhibitor, EP1, EP4, and FP receptors agonists, or calcium channel modulator.
Embodiment 110 is the ophthalmological composition according to embodiment 108, in specific, wherein the miotic agent is alpha-1 adrenergic receptor antagonist.
Embodiment 111 is the ophthalmological composition according to embodiment 108, wherein the mitoic agent is selected from the group consisting of dapiprazole, thymoxamine, brimonidine, nicotine, apraclonidin, bunazosin, phentolamine, and corynathine.
Embodiment 112 is An ophthalmological composition to treat presbyopia comprising a cholinesterase inhibitor according to embodiment 105 in combination with a mitoic agent according to embodiment 107, to act synergistically to increase the depth of the focus of the eye.
Embodiment 113 is the ophthalmological composition according to any one of claims 106-112 wherein the cholinesterase inhibitor concentration ranges from 0.001%-2%, and the to miotic agent concentration ranges from 0.001%-5%.
Embodiment 114 is the ophthalmological composition according to any one of claims 106-113, further comprising a viscosity enhancer and a surfactant selected from the group consisting of an anionic surfactant, a nonionic surfactant, and a combination thereof.
Embodiment 115 is the ophthalmological composition according to embodiment 114 wherein:

the viscosity enhancer is carboxymethyl cellulose or hydroxypropylmethyl cellulose;

the anionic surfactant is selected from the group consisting of a gamma cyclodextrin, sulfobutylether β-cyclodextrin, sodium lauryl sulfate and sodium ester lauryl sulfate;

and the nonionic surfactant is selected from the group consisting of a poloxamer, a polysorbate, sorbitan monolaurate, sorbitan monopalmitate, sorbitan monostearate, sorbitan monooleate, polyoxyl stearate, a polyoxyl alkyl, a cyclodextrin and derivatives thereof.

Embodiment 116 is the ophthalmological composition according to any one of claims 106-115, further comprising sulfobutylether β-cyclodextrin, polyoxyl 40 stearate, or 2-hydroxypropyl beta-cyclodextrin.
Embodiment 117 is the ophthalmological composition according to any one of claims 106-115, further comprising an antioxidant is selected from the group consisting ascorbic acid, glutathione, acetylcysteine, monothioglycerol, metabisulfite salts, sulfurous acid salts (bisulfite), dithiotheitol (DTT), dithiobutylamine (DTBA), and combinations thereof.
Embodiment 118 is the ophthalmological composition according to any one of claims 106-117, further comprising a chelating agent is selected from the group consisting of ethylenediaminetetraacetic acid (EDTA), calcium disodium EDTA, disodium EDTA, pentetic acid (DTPA), and combinations thereof.

The invention is further described in the following examples, which do not limit the scope of the invention described in the claims.

### EXAMPLES

### Example 1

Various Formulations are Tested for Efficacy.

Formulations

A base solution is formulated as follows: Sodium Chloride 0.3%; Edetate Disodium 0.1%; Boric Acid 1.0%; Benzalkonium Chloride 0.01% Sodium Hydroxide (adjust to pH 7.0) and Water. Cholinesterase inhibitor and miotic agent, at a certain concentration, is added to the base solution.

Method

Cholinesterase inhibitor and miotic agent, at certain concentrations, are added to the base solution. The above formulation is administered to the eye of a human adult with presbyopia, shown by his/her discomfort when reading, or inability to read fine print in close distance. Vision is improved after administration of the eye drops. This clinical study is designed to evaluate the effect of a few cholinesterase inhibitors such as neostigmine mesylsulfate, physostigmine sulfate, demecarium bromide, diisopropyl fluorophosphates, echothiophate iodide, alone or together with miotic agent such as dapiprazole, thymoxamine, bunazosin, terazosin, apraclonidine, brimonidine, phentolamin, from 0.001% to less than 2% by weight values on the refractive status of the eye. Subjects with full distance correction are each administered one of the formulations. Each patient has an extensive ocular examination prior to treatment that includes; 1) refractive power of each eye; 2) the unaided distance visual acuity (UDVA) measured using an Snellen chart; 3) the unaided near vision (UNVA) measured at 40 centimeters using a hand-held Rosembaum chart and the Jaeger notation; and 4) the pupillary diameter measured in medium lighting (mesopic) conditions with a special infrared camera device. At baseline prior to installation of ophthalmic solutions, 60, 120, and 360 minute post installation ophthalmic solutions, the same parameters are measured in the right eye and in the left eye. The selected ophthalmic solution are evaluated for longer term treatment.

The following formulations 1-5 are tested for the efficacy of the cholinesterase inhibitors:

| Formulation # | 1 | 2 | 3 | 4 | 5 |
|---|---|---|---|---|---|
| Neostigmine mesylsulfate | 3.00% | | | | |
| Physostigmine sulfate | | 0.25% | | | |
| Demecarium bromide | | | 0.125% | | |
| Diisopropyl | | | | 0.01% | |
| fluorophosphate | | | | | |
| Echothiophate iodide | | | | | 0.03% |
| Sodium Chloride | 0.30% | 0.30% | 0.30% | 0.30% | 0.30% |
| Edetate Disodium | 0.10% | 0.10% | 0.10% | 0.10% | 0.10% |
| Boric Acid | 1.00% | 1.00% | 1.00% | 1.00% | 1.00% |
| Glutathione | 0.50% | 0.50% | 0.50% | 0.50% | 0.50% |
| Benzalkonium Chloride | 0.01% | 0.01% | 0.01% | 0.01% | 0.01% |
| Sodium Hydroxide | adj pH = 7.0 | adj pH = 7.0 | adj pH = 7.0 | adj pH = 7.0 | adj pH = 7.0 |
| Water | qs | qs | qs | qs | qs |

The following formulations 6-10 are tested for the efficacy of the miotic agents:

| Formulation # | 6 | 7 | 8 | 9 | 10 |
|---|---|---|---|---|---|
| Dapiprazole | 0.50% | | | | |
| Thymoxamine | | 0.50% | | | |
| Brimonidine | | | 0.20% | | |
| Phentolamine | | | | 0.80% | |
| Apraclonidin | | | | | 0.50% |
| Sodium Chloride | 0.30% | 0.30% | 0.30% | 0.30% | 0.30% |
| Edetate Disodium | 0.10% | 0.10% | 0.10% | 0.10% | 0.10% |
| Boric Acid | 1.00% | 1.00% | 1.00% | 1.00% | 1.00% |
| Benzalkonium Chloride | 0.01% | 0.01% | 0.01% | 0.01% | 0.01% |
| Sodium Hydroxide | adj pH = 7.0 | adj pH = 7.0 | adj pH = 7.0 | adj pH = 7.0 | adj pH = 7.0 |
| Water | qs | qs | qs | qs | qs |

### Example 2. Clinical Study

Methods

A prospective, double-masked, randomized, controlled clinical study was conducted. Presbyopic subjects over 45 years of age without other ocular diseases and drug allergy were eligible for inclusion. Each eye drops were tested with 3-6 subjects. Bilateral eyes of subjects received topically 4 doses (0.25, 0.75 and 1.5%) of Compound 1 (e.g., cmp #1) eye drops, 3 doses (0.05, 0.083 and 0.25%) of Compound 2 eye drops, 0.25% Compound 3 eye drops, 0.05% Compound 4 eye drops and vehicle eye drops, respectively. The subjects' pupil sizes, intraocular pressure, distance corrected near visual acuity (DCNAV) and ocular tolerance were evaluated pre- and post-treatment at 0.5, 2, and 6 h.

| composition | 1 | 2 | 3 | 4 | 5 | 6 | 7 | 8 |
|---|---|---|---|---|---|---|---|---|
| cmp#1 -01 neostigmine | 0.25% | | | | | | | |
| cmp#1 -02 neostigmine | | 0.75% | | | | | | |
| cmp#1 -03 neostigmine | | | 1.50% | | | | | |
| cmp#2 -01 physostigmine | | | | 0.25% | | | | |
| cmp#2 -02 physostigmine | | | | | 0.083% | | | |
| cmp#2 -03 physostigmine | | | | | | 0.05% | | |
| cmp#3 -01 Demecarium | | | | | | | 0.25% | |
| bromide | | | | | | | | |
| cmp#4 -01 Diisopropyl | | | | | | | | 0.05% |
| fluorophosphate | | | | | | | | |
| Sodium Chloride | 0.30% | 0.30% | 0.30% | 0.30% | 0.30% | 0.30% | 0.30% | 0.30% |
| EDTA Edetate Disodium | 0.10% | 0.10% | 0.10% | 0.10% | 0.10% | 0.10% | 0.10% | 0.10% |
| Boric Acid | 1.00% | 1.00% | 1.00% | 1.00% | 1.00% | 1.00% | 1.00% | 1.00% |
| Benzalkonium Chloride | 0.01% | 0.01% | 0.01% | 0.01% | 0.01% | 0.01% | 0.01% | 0.01% |
| Sodium Hydroxide | adj | adj | adj | adj | adj | adj | adj | adj |
| | pH 7.0 | pH 7.0 | pH 7.0 | pH 7.0 | pH 7.0 | pH 7.0 | pH 7.0 | pH 7.0 |
| Water | qs | qs | qs | qs | qs | qs | qs | qs |

### Results

0.25% compound 1 eye drops had no obvious eye irritation and systemic discomfort. After administering 0.75% compound 1 eye drops for 15 min, two-thirds of the subjects had slight eyelid and eyeball twitching, no obvious eye irritation and systemic discomfort, and the symptoms disappeared in 30 min. After administering 1.25% compound 1 eye drops for 5-15 min, there were increased times of blinking and eyelid and eyeball twitching, and the symptoms gradually alleviated in 30 min and disappeared in 2 h. One third of the subjects had no tolerance problems.

After using 0.05% compound 2 eye drops for 30 min, eye mobility was slightly inflexible in half subjects, lasting about 3-4 hours. After using 0.083% compound 2 eye drops, eye irritation occurred immediately, followed by itching, but it was tolerable, and the eye irritation disappeared in 10 min. Eye twitching occurred 15 min later and lasted 15 min. The upper eyelid and the eyeball were adhered tightly, and the mobility of the upper eyelid and eyeball was slightly weakened. Eye irritation occurred immediately after using 0.25% compound 2 eye drops, eyelid and eyeball twitching occurred 15 min later, lasting 15 min, followed by headache, lasting 1-2 hours.

After administrating 0.25% compound 3 eye drops, half of the subjects had no eye discomfort. Half of the subjects began to suffer from eye discomfort and conjunctival congestion after several hours, lasting for 2-3 days.

Several hours after the administration of 0.05% compound 4 eye drops, subjects developed eye discomfort, itching and conjunctival congestion for about 3 days.

### Efficacy

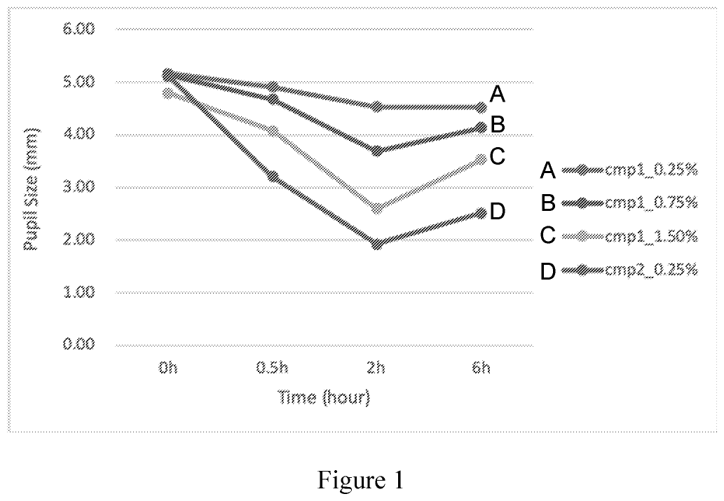

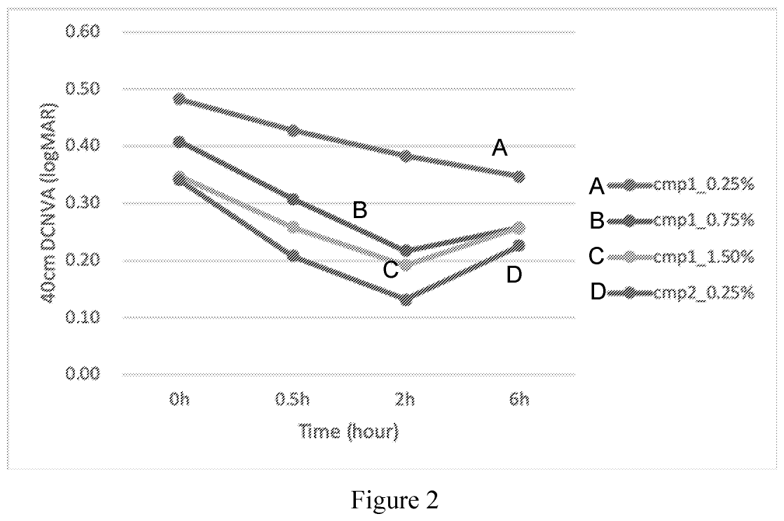

Compound 2 eye drops showed significant miosis (FIG. 1) and improved the distance corrected near visual acuity (FIG. 2) at the concentrations of 0.25% to 1.5%. The effects of 0.25% compound 2 eye drops on miosis and improvement of distance corrected near visual acuity is more than 1.5% compound 2 eye drops. The efficacy of both eye drops reached the highest level in 2 h, then slowly decreased, and was still present after 6 h.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method, the method comprising administering to an eye of a subject, during a treatment period, a cholinesterase inhibitor.

2. The method of claim 1, wherein the method further comprises administering to the eye of the subject, during the treatment period, a miotic agent.

3. The method of claim 1, wherein the cholinesterase inhibitor is selected from the group consisting of physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphates, pharmaceutically acceptable salts thereof, and combinations thereof.

4. The method of claim 3, wherein the cholinesterase inhibitor is selected from the group consisting of physostigmine, neostigmine, demecarium bromide, diisopropyl flurophosphate, pharmaceutically acceptable salts thereof, and combinations thereof.

5. The method of claim 1, wherein the cholinesterase inhibitor is administered at a concentration of about 0.01% to about 2.0% (w/v).

6. The method of claim 2, wherein the miotic agent is selected from the group consisting of muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof.

7. The method of claim 2, wherein the miotic agent is administered at a concentration of about 0.01% to about 2.0% (w/v).

8. The method of claim 1, wherein the eye of the subject is presbyoptic or is at risk for developing presbyopia.

9. The method of claim 1, wherein the amount of the cholinesterase inhibitor, miotic agent, or combination thereof is sufficient to inhibit, slow, or prevent progression of presbyopia in the eye.

10. The method of claim 1, wherein the amount of the cholinesterase inhibitor, miotic agent, or combination thereof is sufficient to achieve a Log MAR visual acuity of less than about 0.3.

11. A composition comprising:
a. a cholinesterase inhibitor; and
b. a pharmaceutically acceptable carrier.

12. The composition of claim 11, further comprising a miotic agent.

13. The composition of claim 11, wherein the cholinesterase inhibitor is selected from the group consisting of physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphates, pharmaceutically acceptable salts thereof, and combinations thereof.

14. The composition of claim 13, wherein the cholinesterase inhibitor is selected from the group consisting of physostigmine, neostigmine, demecarium bromide, diisopropyl flurophosphate, pharmaceutically acceptable salts thereof, and combinations thereof.

15. The composition of claim 11, wherein the cholinesterase inhibitor is present at a concentration of about 0.01% to about 2.0% (w/v).

16. The composition of claim 12, wherein the miotic agent is selected from the group consisting of muscarinic receptor agonists, alpha-1 adrenergic receptor antagonists, alpha-2 adrenergic receptor agonists, β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof.

17. The composition of claim 12, wherein the miotic agent is present at a concentration of about 0.01% to about 2.0% (w/v).

18. The composition of claim 11, wherein the composition is a topical preparation.

19. The composition of claim 18, wherein the composition is a solution, a suspension, an emulsion, a gel, or a sustained release formulation.

20. A method of treating presbyopia, the method comprising administering to an affected eye of a subject in need of such treatment a therapeutically effective amount of a composition of claim 11.

21. (canceled)
